(19) United States
(12) Patent Application Publication
(10) Pub. No.: US 2017/0071522 A1
(43) Pub. Date: Mar. 16, 2017

(54) METHOD OF DIAGNOSING DEPRESSION BY PET IMAGING

(71) Applicants: Ramin PARSEY, East Setauket, NY (US); Christine DELORENZO, Long Island City, NY (US); Gregory SULLIVAN, New York, NY (US)

(72) Inventors: Ramin PARSEY, East Setauket, NY (US); Christine DELORENZO, Long Island City, NY (US); Gregory SULLIVAN, New York, NY (US)

(73) Assignees: THE RESEARCH FOUNDATION FOR THE STATE UNIVERSITY OF NEW YORK, Albany, NY (US); THE TRUSTEES OF COLUMBIA UNIVERSITY IN THE CITY OF NEW YORK, New York, NY (US)

(21) Appl. No.: 15/121,957

(22) PCT Filed: Mar. 5, 2015
(86) PCT No.: PCT/US2015/018992

§ 371 Date: Aug. 26, 2016

Related U.S. Application Data
(60) Provisional application No. 61/949,743, filed on Mar. 7, 2014.

(51) Int. Cl. A61B 5/16; A61K 51/04; A61B 5/055; A61B 5/00; A61B 6/03; A61B 6/00

(52) U.S. Cl. CPC A61B 5/165; A61B 6/037; A61B 6/481; A61B 5/055; A61B 5/0042; A61B 6/501; A61B 6/5217; A61K 51/0459

(57) ABSTRACT

The present invention provides a method of determining whether a subject is afflicted with a depressive disorder comprising:
(i) introducing into the subject a positron emission tomography (PET) radioligand capable of binding with a serotonin 5-HT_{1A} receptor;
(ii) performing one or more PET scans of the subject;
(iii) determining, by analysis of the one or more PET images, a receptor binding potential of the PET radioligand for the serotonin 5-HT_{1A} receptor in a region of interest in the subject;
(iv) comparing the receptor binding potential value of the PET radioligand in the region of interest in the subject to a predetermined receptor binding potential threshold value; and
(v) classifying the subject as having the depressive disorder or as not having the depressive disorder based on the comparison of step (iv), thereby determining whether the subject is afflicted with the depressive disorder.

This application claims priority of U.S. Provisional Application No. 61/949,743, filed Mar. 7, 2014, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

The invention was made with government support under Grant numbers MH40695, MH62185, 1MH074813, and MH090276 awarded by the National Institutes of Health. The government has certain rights in the invention.

### BACKGROUND OF THE INVENTION

Major depressive disorder (MDD) is a highly prevalent psychiatric diagnosis that is associated with a high degree of morbidity and mortality (Marangell et al., 2006; Merikangas et al., 2007; Woods, 2000). This debilitating disorder is currently one of the leading causes of disability nationwide among both medical and psychiatric conditions and is predicted to be the leading cause of disease burden by the year 2030 (Lopez & Murray, 1998; World Health, 2004).

There are currently 945 ways to meet diagnostic criteria for a major depressive episode and thus, patients sharing as few as one common symptom can be diagnosed with major depression. Further, diagnosis of MDD depends on the reliability of current diagnostic classifications and (subjective) structured diagnostic interviews (Karlsson et al., 2010). It is partly for this reason that, for several decades, epidemiological studies reported that women were twice as likely as men to develop MDD, with prevalence rates of 8% and 4%, respectively (Jovanovic et al., 2008; Parker & Brotchie, 2010). Since men and women experience depression differently, these subjective criteria may have led to an under diagnosis of MDD in males. Consistent with this view, a 2013 study reported that when changes in case definitions of MDD were implemented in a way that account for higher rates of anger, aggression and substance abuse in men, MDD prevalence estimates between sexes are eliminated (Martin et al., 2013).

A biomarker is a characteristic that can be objectively measured and used as an indicator of either normal or pathogenic processes (Singh & Rose, 2009). Identification of psychiatric biomarkers for MDD would eliminate the need for subjective diagnosis, and therefore help improve diagnostic classification. Further, such a marker may aid in better classifying the great heterogeneity observed across MDD presentation into more specific sub-diagnostic categories as well as provide much needed evidence of the physiological underpinnings of MDD (Singh & Rose, 2009). Due to its role in MDD, the serotonergic system could give rise to a biomarker of depression. Previous research has implicated the serotonergic system in MDD pathophysiology (Boldrini et al., 2008; Drevets et al., 1999; Parsey et al., 2006; Sargent et al., 2000; Savitz et al., 2009; Stockmeier, 2003) and Selective Serotonin Reuptake Inhibitors (SSRIs) remain the first line MDD treatment, further implicating serotonergic dysfunction in MDD (Blier et al. 1998; G. M. Sullivan et al., 2009). Tools such as Positron Emission Tomography (PET) allow visualization and quantification of serotonin receptor binding in vivo.

PET involves detection of pairs of gamma rays emitted indirectly by a positron-emitting radionuclide (tracer) injected into the body. Images of tracer concentration in the body are then reconstructed by computer analysis. Positron emitting isotopes include carbon, iodine, fluorine, nitrogen, and oxygen. These isotopes can replace their non-radioactive counterparts in target compounds to produce tracers that function biologically and are chemically identical to the original molecules for PET imaging, or can be attached to said counterparts to give close analogues of the respective parent molecule. Among these isotopes, ¹⁸F is a convenient labeling isotope due to its relatively long 109-minute half-life.

### SUMMARY OF THE INVENTION

The present invention provides a method of determining whether a subject is afflicted with a depressive disorder comprising:
(i) introducing into the subject a positron emission tomography (PET) radioligand capable of binding with a serotonin 5-HT_{1A} receptor;
(ii) performing one or more PET scans of the subject;
(iii) determining, by analysis of the one or more PET images, a receptor binding potential of the PET radioligand for the serotonin 5-HT′ A receptor in a region of interest in the subject;
(iv) comparing the receptor binding potential value of the PET radioligand in the region of interest in the subject to a predetermined receptor binding potential threshold value; and
(v) classifying the subject as having the depressive disorder or as not having the depressive disorder based on the comparison of step (iv), thereby determining whether the subject is afflicted with the depressive disorder.

The present invention also provides a method of preparing a report classifying a subject as having a depressive disorder or as not having a depressive disorder which comprises:
(i) receiving the data of one or more PET scans of the subject performed by a PET imaging device after a PET radioligand for a serotonin 5-HT_{1A} receptor was introduced into the subject;
(ii) processing the data to determine a receptor binding potential of the PET radioligand for the serotonin 5-HT_{1A} receptor in a region of interest in the subject and comparing the receptor binding potential value to a predetermined receptor binding potential threshold value; and
(iii) populating a report classifying the subject.

The present invention further provides a method of treating a subject afflicted with a depressive disorder, comprising
(a) determining whether the subject is afflicted with the depressive disorder comprising:
   (i) introducing into the subject a positron emission tomography (PET) radioligand capable of binding with a serotonin 5-HT_{1A} receptor;
   (ii) performing one or more PET scans of the subject;
   (iii) determining, by analysis of the one or more PET images, a receptor binding potential of the PET radioligand for the serotonin 5-HT_{1A} receptor in a region of interest in the subject;
   (iv) comparing the receptor binding potential value of the radioligand in the region of interest in the subject to a predetermined receptor binding potential threshold value; and
   (v) classifying the subject as afflicted with the depressive disorder when the receptor binding potential value of the radioligand in the subject is greater than the predetermined diagnostic threshold value; and
(b) treating the subject based on the determination obtained in step (a).

### BRIEF DESCRIPTION OF THE FIGURES

FIG. 1: [¹¹C]WAY-100635 binding potential (BP_{F}) estimates for the 5-HT_{1A} receptor in male control and male MDD subjects in the raphe nuclei. The horizontal dotted line represents a threshold value that can separate control subjects from MDD subjects. This threshold was used to calculate the sensitivity and specificity of diagnosis. Diamonds or squares represent single measurements of raphe BP_{F} in control and MDD subjects, respectively. Thin capped vertical error bars represent standard errors computed using a bootstrap algorithm that takes into account errors in metabolite, plasma, and image data. Weighted group mean and standard error of the weighted mean of BP_{F} are represented by thick horizontal lines and thick vertical lines, respectively. BPF, binding potential; [¹¹C]WAY-100635, N-(2-(4-(2-methoxyphenyl)-1-piperazinyl)ethyl)-N-(2-pyridinyl)cyclo-hexanecarboxamide; 5-HT_{1A}, serotonin-1A receptor.

FIG. 2: [¹¹C]WAY-100635 binding potential (BP_{F}) estimates for the 5-HT_{1A} receptor in male control, male MDD, male HRO and male remitted subjects in the raphe nuclei. Diamonds, squares, triangles and x's represent single measurements of raphe BP_{F} in control, MDD, HRO and remitted subjects, respectively. Thin capped vertical error bars represent standard errors computed using a bootstrap algorithm that takes into account errors in metabolite, plasma, and image data. Weighted group mean and standard error of the weighted mean of BP_{F} are represented by thick horizontal lines and thick vertical lines, respectively. BPF, binding potential; [¹¹C]WAY-100635, N-(2-(4-(2-methoxyphenyl)-1-piperazinyl)ethyl)-N-(2-pyridinyl)cyclo-hexanecarboxamide; MDD, Major Depressive Disorder; HRO, High Risk Offspring (men who have never had depression themselves, but have at least one parent diagnosed with MDD); Remitted, previously depressed men who have remitted from a major depressive episode; 5-HT_{1A}, serotonin-1A receptor.

### DETAILED DESCRIPTION OF THE INVENTION

The present invention provides a method of determining whether a subject is afflicted with a depressive disorder comprising:
(i) introducing into the subject a positron emission tomography (PET) radioligand capable of binding with a serotonin 5-HT_{1A} receptor;
(ii) performing one or more PET scans of the subject;
(iii) determining, by analysis of the one or more PET images, a receptor binding potential of the PET radioligand for the serotonin 5-HT_{1A} receptor in a region of interest in the subject;
(iv) comparing the receptor binding potential value of the PET radioligand in the region of interest in the subject to a predetermined receptor binding potential threshold value; and
(v) classifying the subject as having the depressive disorder or as not having the depressive disorder based on the comparison of step (iv), thereby determining whether the subject is afflicted with the depressive disorder.

The present invention also provides a method of preparing a report classifying a subject as having a depressive disorder or as not having a depressive disorder which comprises:
(i) receiving the data of one or more PET scans of the subject performed by a PET imaging device after a PET radioligand for a serotonin 5-HT_{1A} receptor was introduced into the subject;
(ii) processing the data to determine a receptor binding potential of the PET radioligand for the serotonin 5-HT_{1A} receptor in a region of interest in the subject and comparing the receptor binding potential value to a predetermined receptor binding potential threshold value; and
(iii) populating a report classifying the subject.

The present invention further provides a method of treating a subject afflicted with a depressive disorder, comprising
(a) determining whether the subject is afflicted with the depressive disorder comprising:
   (i) introducing into the subject a positron emission tomography (PET) radioligand capable of binding with a serotonin 5-HT_{1A} receptor;
   (ii) performing one or more PET scans of the subject;
   (iii) determining, by analysis of the one or more PET images, a receptor binding potential of the PET radioligand for the serotonin 5-HT_{1A} receptor in a region of interest in the subject;
   (iv) comparing the receptor binding potential value of the PET radioligand in the region of interest in the subject to a predetermined receptor binding potential threshold value; and
   (v) classifying the subject as afflicted with the depressive disorder when the receptor binding potential value of the PET radioligand in the subject is greater than the predetermined diagnostic threshold value; and
(b) treating the subject based on the determination obtained in step (a).

In some embodiments, the PET radioligand is introduced by injection into the bloodstream of the subject.

In some embodiments, the analysis of the one or more PET images in step (iii) is a computer analysis.

In some embodiments, the step (ii) further comprises carrying out one or more MRI scans of the subject.

In some embodiments, the MRI images are analyzed to define the boundaries of the region of interest.

In some embodiments, the MRI images are analyzed to define the boundaries of some of the region of interest.

In some embodiments, the subject is classified as having the depressive disorder when the receptor binding potential value of the PET radioligand in the region of interest in the subject is greater than the predetermined diagnostic threshold value.

In some embodiments, the subject is classified as not having the depressive disorder when the receptor binding potential value of the PET radioligand in the region of interest in the subject is about the same or less than the predetermined diagnostic threshold value.

In some embodiments, the PET radioligand contains a radioisotope selected from the group consisting of ³H, ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁴⁴Sc, ⁶⁷Ga, and ⁶⁸Ga.

In some embodiments, the PET radioligand contains a ¹¹C radioisotope. In some embodiments, the PET radioligand contains a ¹⁸F radioisotope.

In some embodiments, the PET radioligand is radiolabeled N-[2-[4-(2-methoxyphenyl)-1-piperazinyl]ethyl]-N-2-pyridinylcyclohexanecarboxamide.

In some embodiments, the radiolabeled N-[2-[4-(2-methoxyphenyl)-1-piperazinyl]ethyl]-N-2-pyridinylcyclohexanecarboxamide is radiolabeled with carbon-11.

In some embodiments, the radiolabeled N-[2-[4-(2-methoxyphenyl)-1-piperazinyl]ethyl]-N-2-pyridinylcyclohexanecarboxamide is radiolabeled with carbon-11 at the carbonyl carbon.

In some embodiments, the radiolabeled N-[2-[4-(2-methoxyphenyl)-1-piperazinyl]ethyl]-N-2-pyridinylcyclohexanecarboxamide is radiolabeled with carbon-11 at the methyl carbon of the methoxy group.

In some embodiments, the region of interest is in the brain.

In some embodiments, the region of interest is selected from the group consisting of the raphe nucleus, dorsolateral prefrontal cortex, medial prefrontal cortex, orbito-frontal cortex, anterior cingulate cortex, subgenual prefrontal cortex, temporal cortex, parietal cortex, occipital cortex, amygdala, uncus, hippocampal formation, entorhinal cortex, parahippocampal gyrus, insula, dorsal raphe nuclei, and cerebellum.

In some embodiments, the region of interest is the dorsal raphe nuclei.

In some embodiments, the region of interest is the raphe nuclei.

In some embodiments, the methods wherein the predetermined receptor binding potential threshold value is 30.0. In some embodiments, the methods wherein the predetermined receptor binding potential threshold value is 32.0. In some embodiments, the methods wherein the predetermined receptor binding potential threshold value is 34.0. In some embodiments, the methods wherein the predetermined receptor binding potential threshold value is 36.0. In some embodiments, the methods wherein the predetermined receptor binding potential threshold value is 38.0. In some embodiments, the methods wherein the predetermined receptor binding potential threshold value is 40.0. In some embodiments, the methods wherein the predetermined receptor binding potential threshold value is 42.0. In some embodiments, the methods wherein the predetermined receptor binding potential threshold value is 44.0. In some embodiments, the methods wherein the predetermined receptor binding potential threshold value is 46.0. In some embodiments, the methods wherein the predetermined receptor binding potential threshold value is 48.0. In some embodiments, the methods wherein the predetermined receptor binding potential threshold value is 50.0.

In some embodiments, the methods wherein the predetermined receptor binding potential threshold value is 39.9.

In some embodiments, the depressive disorder is major depression.

In some embodiments, the subject is a human subject.

In some embodiments, the human subject is a male subject.

In some embodiments, the human subject is a female subject.

In some embodiments, the subject had never undergone antidepressant treatment. In some embodiments, the subject had gone without antidepressant treatment for at least four years.

In some embodiments, the male subject had never undergone antidepressant treatment. In some embodiments, the male subject had gone without antidepressant treatment for at least four years.

In some embodiments, the subject is treated with an anti-depressant. In some embodiments, the male subject is treated with an anti-depressant.

In some embodiments, the anti-depressant is selected from the group consisting of Citalopram, Escitalopram, Paroxetine, Fluoxetine, Fluvoxamine, Sertraline, Desvenlafaxine, Duloxetine, Levomilnacipran, Milnacipran, Venlafaxine, Tramadol, Sibutramine, Etoperidone, Lubazodone, Nefazodone, Trazodone, Atomoxetine, Reboxetine, Viloxazine, Bupropion, Amphetamine, Dextroamphetamine, Dextromethamphetamine, Lisdexamfetamine, Amitriptyline, Butriptyline, Clomipramine, Desipramine, Dosulepin, Doxepin, Imipramine, Iprindole, Lofepramine, Melitracen, Nortriptyline, Opipramol, Protriptyline, Trimipramine, Amoxapine, Maprotiline, Mianserin, Mirtazapine, Isocarboxazid, Phenelzine, Selegiline, Tranylcypromine, Moclobemide, Pirlindole, Mianserin, Mirtazapine, Vilazodone, Vortioxetine, Tandospirone, Quetiapine, and AZD6765.

In some embodiments, the subject is treated with psychotherapy. In some embodiments, the male subject is treated with psychotherapy.

In some embodiments, the psychotherapy is selected from the group consisting of Psychodynamic Therapy, Interpersonal Therapy and Cognitive Behavioral Therapy.

The present invention provides a method of determining whether a male subject is afflicted with a depressive disorder comprising:
(i) introducing into the male subject a positron emission tomography (PET) radioligand capable of binding with a serotonin 5-HT_{1A} receptor;
(ii) performing one or more PET scans of the male subject;
(iii) determining, by analysis of the one or more PET images, a receptor binding potential of the PET radioligand for the serotonin 5-HT_{1A} receptor in a region of interest in the male subject;
(iv) comparing the receptor binding potential value of the PET radioligand in the region of interest in the male subject to a predetermined receptor binding potential threshold value; and
(v) classifying the male subject as having the depressive disorder or as not having the depressive disorder based on the comparison of step (iv), thereby determining whether the male subject is afflicted with the depressive disorder.

The present invention also provides a method of preparing a report classifying a male subject as having a depressive disorder or as not having a depressive disorder which comprises:
(i) receiving the data of one or more PET scans of the male subject performed by a PET imaging device after a PET radioligand for a serotonin 5-HT_{1A} receptor was introduced into the male subject;
(ii) processing the data to determine a receptor binding potential of the PET radioligand for the serotonin 5-HT_{1A} receptor in a region of interest in the male subject and comparing the receptor binding potential value to a predetermined receptor binding potential threshold value; and
(iii) populating a report classifying the male subject.

The present invention further provides a method of treating a male subject afflicted with a depressive disorder, comprising
(a) determining whether the male subject is afflicted with the depressive disorder comprising:
   (i) introducing into the male subject a positron emission tomography (PET) radioligand capable of binding with a serotonin 5-HT_{1A} receptor;
   (ii) performing one or more PET scans of the male subject;
   (iii) determining, by analysis of the one or more PET images, a receptor binding potential of the PET radioligand for the serotonin 5-HT_{1A} receptor in a region of interest in the male subject;
   (iv) comparing the receptor binding potential value of the PET radioligand in the region of interest in the male subject to a predetermined receptor binding potential threshold value; and
   (v) classifying the subject as afflicted with the depressive disorder when the receptor binding potential value of the radioligand in the male subject is greater than the predetermined diagnostic threshold value; and
(b) treating the male subject based on the determination obtained in step (a).

In some embodiments, the PET radioligand is introduced by injection into the bloodstream of the male subject.

In some embodiments, the analysis of the one or more PET images in step (iii) is a computer analysis.

In some embodiments, step (ii) further comprises carrying out one or more MRI scans of the subject.

In some embodiments, the MRI images are analyzed to define the boundaries of the region of interest.

In some embodiments, the MRI images are analyzed to define the boundaries of some of the region of interest.

In some embodiments, the male subject is classified as having the depressive disorder when the receptor binding potential value of the PET radioligand in the region of interest in male subject is greater than the predetermined diagnostic threshold value.

In some embodiments, the male subject is classified as not having the depressive disorder when the receptor binding potential value of the radioligand in the region of interest in the male subject is about the same or less than the predetermined diagnostic threshold value.

In some embodiments, the PET radioligand contains a radioisotope selected from the group consisting of ³H, ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁴⁴Sc ⁶⁷Ga, and ⁶⁸Ga.

In some embodiments, the PET radioligand contains a ¹¹C radioisotope.

In some embodiments, the PET radioligand contains a F radioisotope.

In some embodiments, the PET radioligand is radiolabeled N-[2-[4-(2-methoxyphenyl)-1-piperazinyl]ethyl]-N-2-pyridinylcyclohexanecarboxamide.

In some embodiments, the radiolabeled N-[2-[4-(2-methoxyphenyl)-1-piperazinyl]ethyl]-N-2-pyridinylcyclohexanecarboxamide is radiolabeled with carbon-11.

In some embodiments, the radiolabeled N-[2-[4-(2-methoxyphenyl)-1-piperazinyl]ethyl]-N-2-pyridinylcyclohexanecarboxamide is radiolabeled with carbon-11 at the carbonyl carbon.

In some embodiments, the radiolabeled N-[2-[4-(2-methoxyphenyl)-1-piperazinyl]ethyl]-N-2-pyridinylcyclohexanecarboxamide is radiolabeled with carbon-11 at the methyl carbon of the methoxy group.

In some embodiments, the PET radioligand is radiolabeled 2-(4-(4-(2-methoxyphenyl)piperazin-1-yl)butyl)-4-methyl-1,2,4-triazine-3,5 (2H,4H) dione (¹¹C-CUMI).

In some embodiments, the radiolabeled 2-(4-(4-(2-methoxyphenyl)piperazin-1-yl)butyl)-4-methyl-1,2,4-triazine-3,5 (2H,4H) dione is radiolabeled with carbon-11.

In some embodiments, the radiolabeled 2-(4-(4-(2-methoxyphenyl)piperazin-1-yl)butyl)-4-methyl-1,2,4-triazine-3,5 (2H,4H) dione is radiolabeled with carbon-11 at the methoxy carbon.

In some embodiments, the region of interest is in the brain.

In some embodiments, the region of interest is selected from the group consisting of the raphe nucleus, dorsolateral prefrontal cortex, medial prefrontal cortex, orbito-frontal cortex, anterior cingulate cortex, subgenual prefrontal cortex, temporal cortex, parietal cortex, occipital cortex, amygdala, uncus, hippocampal formation, entorhinal cortex, parahippocampal gyrus, insula, dorsal raphe nuclei, and cerebellum.

In some embodiments, the region of interest is the dorsal raphe nuclei.

In some embodiments, the region of interest is the raphe nuclei.

In some embodiments, the predetermined receptor binding potential threshold value is 30.0. In some embodiments, the methods wherein the predetermined receptor binding potential threshold value is 32.0. In some embodiments, the predetermined receptor binding potential threshold value is 34.0. In some embodiments, the predetermined receptor binding potential threshold value is 36.0. In some embodiments, the predetermined receptor binding potential threshold value is 38.0. In some embodiments, the predetermined receptor binding potential threshold value is 40.0. In some embodiments, the predetermined receptor binding potential threshold value is 42.0. In some embodiments, the predetermined receptor binding potential threshold value is 44.0. In some embodiments, the predetermined receptor binding potential threshold value is 46.0. In some embodiments, the predetermined receptor binding potential threshold value is 48.0. In some embodiments, the predetermined receptor binding potential threshold value is 50.0.

In some embodiments, the predetermined receptor binding potential threshold value is 39.9.

In some embodiments, the depressive disorder is major depression.

In some embodiments, the human male subject had never undergone antidepressant treatment.

In some embodiments, the human male subject had gone without antidepressant treatment for at least four years.

In some embodiments of the above method, the depressive disorder is major depression.

In some embodiments of the above method, the male subject is treated with an anti-depressant.

In some embodiments of the above method, the male subject is treated with selective serotonin reuptake inhibitors (SSRIs), serotonin and norepinephrine reuptake inhibitors (SNRIs), atypical antidepressants, tricyclic antidepressants, tetracyclic antidepressants, or monoamine oxidase inhibitors (MAOIs).

In some embodiments of the above method, the anti-depressant is selected from the group consisting of Citalopram, Escitalopram, Paroxetine, Fluoxetine, Fluvoxamine, Sertraline, Desvenlafaxine, Duloxetine, Levomilnacipran, Milnacipran, Venlafaxine, Tratnadol, Sibutramine, Etoperidone, Lubazodone, Nefazodone, Trazodone, Atomoxetine, Reboxetine, Viloxazine, Bupropion, Amphetamine, Dextroamphetamine, Dextromethamphetamine, Lisdexamfetamine, Amitriptyline, Butriptyline, Clomipramine, Desipramine, Dosulepin, Doxepin, Imipramine, Iprindole, Lofepramine, Melitracen, Nortriptyline, Opipramol, Protriptyline, Trimipramine, Amoxapine, Maprotiline, Mianserin, Mirtazapine, Isocarboxazid, Phenelzine, Selegiline, Tranylcypromine, Moclobemide, Pirlindole, Mianserin, Mirtazapine, Vilazodone, Vortioxetine, Tandospirone, Quetiapine, and AZD6765.

In some embodiments of the above method, the male subject is treated with psychotherapy. In some embodiments, the psychotherapy is selected from the group consisting of Psychodynamic Therapy, Interpersonal Therapy and Cognitive Behavioral Therapy.

The present invention provides a method of determining whether a subject is at risk for developing a depressive disorder comprising:
(i) introducing into the subject a positron emission tomography (PET) radioligand capable of binding with a serotonin 5-HT_{1A} receptor;
(ii) performing one or more PET scans of the subject;
(iii) determining, by analysis of the one or more PET images, a receptor binding potential of the PET radioligand for the serotonin 5-HT_{1A} receptor in a region of interest in the subject;
(iv) comparing the receptor binding potential value of the PET radioligand in the region of interest in the subject to a predetermined receptor binding potential threshold value; and
(v) classifying the subject as having the depressive disorder or as not having the depressive disorder based on the comparison of step (iv), thereby determining whether the subject is at risk for developing a depressive disorder.

The present invention provides a method of determining whether a male subject is afflicted with a depressive disorder comprising:
(i) introducing into the male subject a positron emission tomography (PET) radioligand capable of binding with a serotonin 5-HT_{1A} receptor in the raphe nuclei in the male subject;
(ii) performing one or more PET scans of the male subject;
(iii) determining, by analysis of the one or more PET images, a receptor binding potential of the PET radioligand for the serotonin 5-HT_{1A} receptor in the raphe nuclei in the male subject;
(iv) comparing the receptor binding potential value of the radioligand in the raphe nuclei in the male subject to a predetermined receptor binding potential threshold value; and
(v) classifying the male subject as having the depressive disorder or as not having the depressive disorder based on the comparison of step (iv), thereby determining whether the male subject is afflicted with the depressive disorder.

In some embodiments of the above method, the predetermined receptor binding potential threshold value radioligand in the raphe nuclei is 30.0, 32.0, 34.0, 36.0, 38.0, 40.0, 42.0, 44.0, 46.0, 48.0, or 50.0.

In some embodiments of the above method, the predetermined receptor binding potential threshold value radioligand in the raphe nuclei is 39.9.

The present invention provides a method of determining whether a subject is at risk for developing a depressive disorder comprising:
(i) introducing into the subject a positron emission tomography (PET) radioligand capable of binding with a serotonin 5-HT_{1A} receptor in the raphe nuclei of the subject;
(ii) performing one or more PET scans of the subject;
(iii) determining, by analysis of the one or more PET images, a receptor binding potential of the PET radioligand for the serotonin 5-HT_{1A} receptor in the raphe nuclei in the subject;
(iv) comparing the receptor binding potential value of the radioligand in the subject to a predetermined receptor binding potential threshold value; and
(v) classifying the subject as at risk for developing the depressive disorder or as not at risk for developing the depressive disorder based on the comparison of step (iv), thereby determining whether the subject is at risk for developing the depressive disorder.

The present invention provides a method of determining whether a male subject is at risk for developing a depressive disorder comprising:
(i) introducing into the male subject a positron emission tomography (PET) radioligand capable of binding with a serotonin 5-HT_{1A} receptor in the raphe nuclei of the male subject;
(ii) performing one or more PET scans of the male subject;
(iii) determining, by analysis of the one or more PET images, a receptor binding potential of the PET radioligand for the serotonin 5-HT_{1A} receptor in the raphe nuclei of the male subject;
(iv) comparing the receptor binding potential value of the radioligand in the male subject to a predetermined receptor binding potential threshold value; and
(v) classifying the male subject as at risk for developing the depressive disorder or as not at risk for developing the depressive disorder based on the comparison of step (iv), thereby determining whether the male subject is at risk for developing the depressive disorder.

In some embodiments of the above method, the predetermined receptor binding potential threshold value radioligand in the raphe nuclei is 30.0, 32.0, 34.0, 36.0, 38.0, 40.0, 42.0, 44.0, 46.0, 48.0, or 50.0.

In some embodiments of the above method, the predetermined receptor binding potential threshold value radioligand in the raphe nuclei is 39.9.

In some embodiments of any of the above methods, the subject is a human male subject.

In some embodiments of any of the above methods, the subject is a human female subject.

In some embodiments of any of the above methods, the depressive disorder is major depression.

In some embodiments of any of the above methods, the one or more PET scans or one or more MRI scans are performed on the region of interest in the subject.

N-[2-[4-(2-Methoxyphenyl)-1-piperazinyl]ethyl]-N-2-pyridinylcyclohexanecarboxamide (WAY-100635) has the following structure and is available for purchase as Catalog No. W108 from Sigma-Aldrich (St. Louis, Mo., USA):

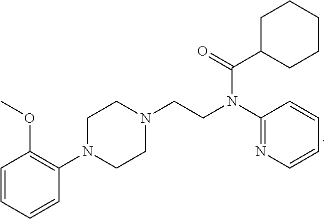 [SMILES: COc1ccccc1N1CCN(CCN(C(=O)C2CCCCC2)c2ccccn2)CC1]

The preparation of [Carbonyl-¹¹C]WAY-100635 is described in Hwang et al., 1999, the contents of which is hereby incorporated by reference.

2-(4-(4-(2-methoxyphenyl)piperazin-1-yl)butyl)-4-methyl-1,2,4-triazine-3,5 (2H,4H) dione (CUMI-101) has the following structure:

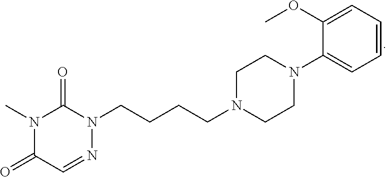 [SMILES: COc1ccccc1N1CCN(CCCCn2ncc(=O)n(C)c2=O)CC1]

In some embodiments, BPF, including raphe BPF, is calculated using the tracer [¹¹C]CUMI-101, a 5-HT_{1A} partial agonist. [¹¹C]CUMI-101 allows for the BPF to be calculated without the need for blood sampling and the insertion of an arterial cannula (Hendry, N. et al., 2011; Milak, M. S. et al., 2008; Milak, M. S. et al., 2010b).

In some embodiments, the tracer is [O-methyl-¹¹C] 2-(4-(4-(2-methoxyphenyl)piperazin-1-yl)butyl)-4-methyl-1,2,4-triazine-3,5 (2H,4H) dione (¹¹C-CUMI).

In some embodiments, BPF, including raphe BP_{F}, is calculated using an ¹⁸F labeled CUMI-101. Due to its 109-minute half-life, an F-18 version of this PET tracer can be shipped to other research centers and does not require a cyclotron on site in order to make the radiotracer.

As used herein, “Receptor Binding Potential”, “Binding Potential” or “BPF” refers to the ratio at equilibrium of the concentration of specifically bound radioligand in tissue to the concentration of free radioligand in tissue.

Binding potential in a region of interest refers to the ratio at equilibrium of the concentration of specifically bound radioligand in tissue of the region of interest to the concentration of free radioligand in tissue (Innis et al, 2007).

In some embodiments, the radioligand binds to serotonin 5-HT_{1A} receptors in the tissue of the region of interest.

As used herein, “predetermined receptor binding potential threshold value” refers to a threshold value of receptor binding potential of a PET radioligand to serotonin 5-HT_{1A} receptors in a region of interest.

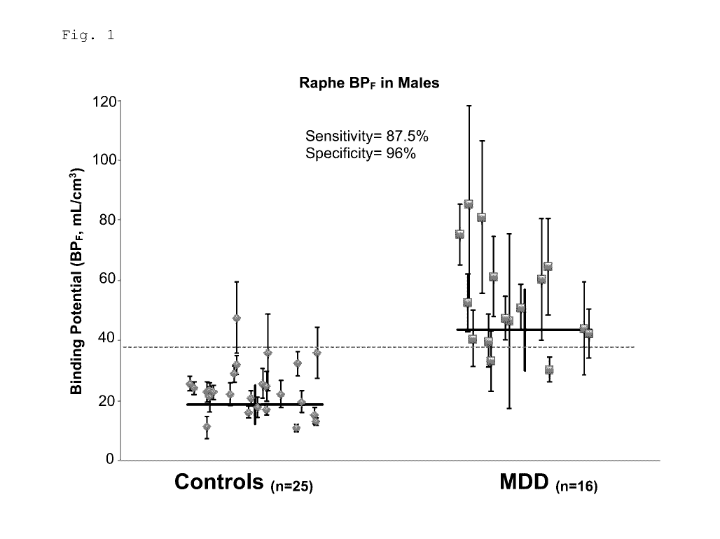

In some embodiments, the “predetermined receptor binding potential threshold value” refers to a threshold value of receptor binding potential of a PET radioligand to serotonin 5-HT_{1A} receptors in the raphe (the region from which all serotonergic neurons originate). This threshold is indicated by the green dotted line in FIG. 1.

In order to determine the proper threshold for diagnostic classification, the diagnostic sensitivity and specificity associated with using each measured raphe BP_{F} value as the diagnostic cutoff were calculated. The minimum value of a cost function consisting of the (negative) sum of these sensitivity and specificity measures was sought. The threshold of 39.9 mL/cm³ is a solution that minimized this cost function. Due to the separation of subjects at this level of binding, the threshold can be determined to within 3 mL/cm³.

In some embodiments, the predetermined receptor binding potential threshold value is determined by analyzing a male control subject or group of male control subjects that are not afflicted with a depressive disorder.

In some embodiments, the predetermined receptor binding potential threshold value is determined by analyzing a male control subject or group of male control subjects that have not been diagnosed with a depressive disorder.

In some embodiments, the predetermined receptor binding potential threshold value is determined by analyzing a male control subject or group of male control subjects that have been diagnosed as not having the depressive disorder.

In some embodiments, the free fraction (f_{P}) of a PET tracer, measured from a single venous sample is used in the calculation of BP_{F} (Milak, M. S. et al., 2010a). The f_{P} value for [Carbonyl-¹¹C]WAY-100635 is directly correlated with 5-HT_{1A} BP_{F} in the raphe nucleus (RN) and [Carbonyl-¹¹C]WAY-100635 acts as a surrogate biomarker for MDD. With a direct correlation present, simple venous sampling following administration of [Carbonyl-¹¹C]WAY-100635 allows for MDD diagnosis.

In the present application, all numbers disclosed herein may vary by 1 percent, 2 percent, 5 percent, or up to 20 percent if the word “about” is used in connection therewith. This variation may be applied to all numbers disclosed herein.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### Materials and Methods

### Participants

This study was approved by the Institutional Review Boards of the New York State Psychiatric Institute and Columbia University Medical Center. 107 subjects between the ages of 18 and 65 were evaluated in this study. These subjects were a combined cohort from three previously reported studies (Miller et al., 2009; Parsey et al., 2010; Parsey et al., 2006) as well as 11 additional subjects. Fifty subjects met DSM (Diagnostic and Statistical Manual of Mental Disorders) IV criteria for Major Depressive Disorder (34 female, 16 male) and fifty-seven were healthy volunteers (32 female, 25 male). All participants provided written informed consent after learning the description of the study protocol. All diagnoses were agreed upon by at least three senior psychiatrists. All of the MDD subjects were categorized as Not Recently Medicated (NRM): defined as greater than 4 years since antidepressant treatment. Study criteria for depressed subjects included: 1) age 18 to 65 years; 2) DSM IV criteria for current MDD; 3) absence of any psychotropic medications for at least 2 weeks (4 years for antidepressants, 4 weeks for neuroleptics), except benzodiazepines, which were discontinued three days prior to the scan; 4) absence of lifetime history of alcohol or substance abuse or dependence; 5) absence of life-time exposure to 3,4-methylenedioxymethamphetamine (MDMA, “ecstasy”); 6) absence of significant medical conditions; 7) absence of pregnancy; 8) capacity to provide informed consent; and 9) absence of psychosis, bipolar disorder, or schizophrenia. Screening was performed via physical exam, history, routine blood and urine tests.

### Clinical Assessments

The HDRS-17 (Hamilton, 1960), the Beck Inventory (BDI) (Beck et al., 1961), and the Global Assessment Scale (Endicott et al., 1976) were utilized to assess depression severity and functional impairment.

### Radiochemistry and Input Function Measures

Measurements of arterial input function, metabolites and free plasma fraction (fₚ) were also made as described previously (Parsey et al., 2005; Parsey et al., 2000).

### Genotyping

The functional 5-HT_{1A} gene promoter region single nucleotide polymorphism (SNP) known as 5-HT_{1A} C(-1019)G was genotyped for a bi-allelic classification i.e. CC, CG or GG, for each participant as previously described (Huang et al., 2004; Wu & Comings, 1999).

### PET Acquisition

Imaging was performed as described Parsey et al., 2000, the contents of which is hereby incorporated by reference. Briefly, after an Allen test and subcutaneous administration of 2% lidocaine, a catheter was inserted in the radial artery. A venous catheter was also inserted into a forearm vein on the opposite side for PET tracer administration. Head movement was minimized with a polyurethane head immobilizer system (Soule Medical, Tampa, Fla., USA), molded around the head of the subject. PET imaging was performed with the ECAT EXACT HR+(Siemens/CTI, Knoxville, Tenn., USA) (63 slices covering an axial field of view of 15.5 cm, axial sampling of 2.46 mm, in 3D mode. A 10-min transmission scan was acquired before injection. After injection of [¹¹C]WAY-100635, as an intravenous bolus over 45 secs using an injection pump, emission data were collected for 110 mins as 20 successive frames of increasing duration (3×20 secs, 3×1 min, 3×2 mins, 2×5 mins, 9×10 mins). Images were reconstructed using the 3D-RP algorithm implemented on a vector processor (CTI, Knoxville, Tenn., USA) to a 128×128 matrix (pixel size of 1.7×1.7 mm²) with attenuation correction and a Shepp 0.5 filter (cutoff 0.5 cycles/projection rays) resulting in an in-plane and axial resolution (i.e. full width half-maximum) of 4.4 mm and 4.1 mm in air and at the center of the field of view (Brix et a.l, 1997). Scatter correction was performed using the technique implemented by the manufacturer (Watson et al., 1995).

### Input Function Measurement

Input function and measurement of metabolites were conducted as described previously (Parsey et al., 2000). Briefly, after radiotracer injection, 30 arterial samples were collected every 5 secs with an automated sampling system for the first 2 mins, and manually thereafter at longer intervals. After centrifugation (10 mins at 3800 g), plasma was collected in 200-4 aliquots and radioactivity was counted in a gamma counter (Wallac 1480 Wizard 3 M Automatic Gamma Counter). Five samples were processed to measure the fraction of unmetabolized [¹¹C]WAY-100635 by high-pressure liquid chromatography (HPLC). The five measured unmetabolized [¹¹C]WAY-100635 fractions were fit with the Hill function (Gunn et al, 1998). The input function was the product of total counts and interpolated unmetabolized [¹¹C]WAY-100635 fraction. The measured input fountain values (Cₐ(t), μCi/mL) were fit to a straight line from time zero to the peak followed by the sum of three exponentials after the peak. The fitted values were used as input to the kinetic analysis. For the determination of the plasma free fraction (fₚ), triplicate 200-μL aliquots of plasma collected before injection were mixed with a radiotracer, pipetted into ultrafiltration units (Centrifree, Amicon, Danvers, Mass., USA) and centrifuged at room temperature (20 mins at 3800 g). Plasma and ultrafiltrate activities were then counted, and fₚ was calculated as the ratio of ultrafiltrate to total activity concentrations (Cleare and Bond, 2000).

### MRI Acquisition and Analysis

MRIs were acquired either on a GE 1.5 T or 3.0 T Signa Advantage system. A sagittal scout (localizer) was performed to identify the AC-PC plane (1 min). Regions of interests (ROIs) were labeled on each subject's skull stripped and segmented (into grey/white matter and cerebrospinal fluid) MRI as previously described (Parsey et al., 2000) and included the ventral prefrontal cortex (VPFC), medial prefrontal cortex (MPFC), dorsolateral prefrontal cortex (DLPFC), anterior cingulate cortex (ACN), cingulate (posterior) cortex (CIN), amygdala (AMY), hippocampus (HIP), parahippocampal gyrus (PHG), insular cortex (INS), temporal cortex (TEM), parietal cortex (PAR), and occipital cortex (OCC). Because the boundaries of the median and dorsal raphe nuclei (RN) are not identifiable on MRI, a 2 cm³ ellipsoid was manually placed on the raphe nuclei of each individual's mean PET image, completely encompassing the high [¹¹C]WAY-100635 binding region of the posterior midbrain. The reference region, cerebellar white matter, is a region of cerebellum is virtually devoid of 5-HT_{1A} (Parsey et al., 2005). It was either defined from the automated white matter segmentation in the cerebellum or by manually outlining a circular region on the MRI. For cortical regions, the ROIs were modified, as previously described, to include only gray matter voxels.

### PET Analysis

PET data analyses were performed as described previously (Parsey et al., 2005). Image analysis was performed using MATLAB (The Mathworks, Natick, Mass.) with extensions to the following open source packages: Functional Magnetic Resonance Imaging of the Brain's Linear Image Registration Tool (FLIRT) v5. (Oxford Center for Functional Magnetic Resonance Imaging of the Brain, Oxford, England) (Jenkinson & Smith, 2001), Brain Extraction Tool (BET) v1.2 (Oxford Centre for Functional Magnetic Resonance Imaging of the Brain) (Smith, 2002), and University College of London's Statistical Parametric Mapping (SPM5) (Wellcome Department of Imaging Neuroscience, London, United Kingdom) normalization (Ashburner & Friston, 1999) and segmentation routines (Ashburner & Friston, 2005). To correct for subjection motion during the PET scan, de-noising filter techniques were applied to later PET images. The eighth frame was used as a reference onto which all other frames were aligned using rigid body FLIRT. For co-registration, a mean of the motion-corrected frames was registered using FLIRT to the MRI. Time activity curves were generated by plotting the average regional activity within each co-registered PET frame over the time of the scan.

### Quantitative Analysis

Regional distribution volumes of [¹¹C]WAY-100635 were derived from kinetic analysis using the arterial input function and a two-tissue compartment (2T) model with constrained parameters—K₁/K₂ ratio fixed to that of the cerebellar white matter (see Parsey et al., 2000 for details).

The model included the concentration of tracer in the arterial plasma compartment (C_{P}), free in tissue water (C_{FT}), nonspecifically bound (C_{NS}), and specifically bound compartment (C_{S}).

The equilibrium distribution volume of a compartment i (V_{Ti}, mL/g) was defined as the ratio of the total tracer concentration in this compartment to the free plasma concentration at equilibrium (C_{T}/C_{P}), where C_{T}=C_{S}+C_{NS}+C_{FT}.

V_{ND} is defined as the distribution volume of the nondisplaceable compartment. BP_{F}=(V_{T}−V_{ND})/f_{P} is equal to the ratio of the available receptor density (Bₐᵥₐᵢₗ, nmol/L per g of tissue) and affinity (1/K_{D}, nmol/L per mL of brain water) (Innis et al., 2007).

The contribution of plasma total activity to the regional activity was calculated assuming a 5% blood volume in the ROI and subtracted from the regional activity before analysis. All kinetic parameters were derived by nonlinear regression using a Levenberg-Marquart least-squares minimization procedure implemented in MATLAB (The Math Works, Inc., South Natick, Mass., USA). Given the unequal sampling over time (increasing frame acquisition time from beginning to end of the study), the least-squares minimization procedure was weighted by the square root of the frame acquisition time.

### Statistics

Standard errors (SE) were computed for each estimated BP_{F} value using a bootstrap algorithm that takes into account errors in metabolite, plasma and brain data (Ogden & Tarpey, 2006). ROI-level BP_{F} estimates were natural-log transformed before statistical modeling in order to account for heterogeneity of variances across regions. Linear mixed-effects models with standard errors of transformed ROI-level BP_{F} estimates as weights were fit to the transformed ROI-level BP_{F} estimates with brain region as the fixed effect and the subject as the random effect. The dependency structure for all ROI within the same subject was chosen based on Akaike Information Criterion (AIC). The final structure used had a generalized compound symmetry structure allowing different variance components in different brain regions and fixed correlation in any two brain regions within the same subject. The covariates in the model included brain region, gender, diagnosis group, and the interaction between brain region and gender. There were no other interaction terms in first or higher order that reached statistical significance level. Significance level was set at 0.05 and p-values were reported without multiple comparison adjustment. All tests were two-sided. Model fitting was computed using both SAS 9.2 (SAS Inc., Cary, N.C.) and R 3.0.2 (R Project for Statistical Computing; www.R-project.org).

### Example 1

### Effects of Sex

Consistent with previous studies (Parsey et al., 2006), in controls, males had a 17.8% lower BP_{F} than females (df=103, p=0.0194) across all ROIs. However, in depressed subjects, males had a 14.7% higher BP_{F} than females (df=103, p=0.1481).

When looking at each sex individually, female MDD subjects had 19.7% higher BP_{F} across all regions, compared to female controls (df=103, p=0.0197). As depicted in FIG. 1, Male MDD subjects had 67.0% higher BP_{F} across all regions, compared to male controls (df=103, p<0.0001).

### Example 2

### Raphe Nuclei

Post hoc analysis assessing BP_{F} differences between control and MDD subjects, in each sex, showed that a region by diagnosis interaction was present in each sex and that certain regions exhibit greater BP_{F} separation between controls and MDD subjects than others (Table 1).

As noted in Table 1, the largest separation in binding between MDD and control subjects occurs in the raphe of the males. Due to this significant separation (132%, p=0.000), this region was examined to determine a diagnostic threshold that separated male MDD and control subjects. In order to determine the proper threshold for diagnostic classification, the sensitivity and specificity associated with using each measured raphe BP_{F} value as the diagnostic cutoff were calculated. The minimum value of a cost function consisting of the (negative) sum of these sensitivity and specificity measures was sought. The threshold of 39.9 mL/cm³ is a solution that minimized this cost function. Due to the separation of subjects at this level of binding, the threshold can be determined to within 3 mL/cm₃. Using this threshold, the diagnostic sensitivity is 87.5%, specificity is 96.0% with a positive predictive value of 93.3% and a negative predictive value of 92.3%. Using [Carbonyl-C-11]WAY-100635 f_{P} values only achieves 56% specificity.

TABLE 1
Percent Differences in BP_{F} Between Control and MDD Subjects

| | Male | Female |
|---|---|---|
| RN | 132.59% | 10.68% |
| AMY | 64.07% | 25.03% |
| HIP | 74.22% | 19.59% |
| PIP | 87.96% | 34.76% |
| TEM | 87.07% | 27.23% |
| ACN | 80.47% | 30.75% |
| CIN | 80.79% | 26.65% |
| DOR | 83.69% | 31.10% |
| MED | 84.79% | 31.13% |
| ORB | 84.94% | 28.59% |
| INS | 76.78% | 22.48% |
| OCC | 95.18% | 20.50% |
| PAR | 98.55% | 23.82% |

Although, on average, the female MDD subjects have higher binding than female controls, the separation between diagnostic groups is much smaller (10.7%, p=0.078), and therefore sensitivity and specificity in distinguishing MDD in females is not as high (sensitivity=56%, specificity=75%). However, measuring 5-HT_{1A} binding in females while adjust for covariates such as location of female subjects in their menstrual cycle is useful for the diagnosis of depressive disorders in female subjects. Using venous sampling, fluctuations in estrogen, and progesterone, levels that occur during the menstrual cycle are taken in account to more accurately calculate measures of 5-HT_{1A} binding.

Female gonadal hormone levels throughout the menstrual cycle directly affect radioligand binding to the serotonin 5-HT_{1A} receptor. Binding potentials of female MDD subjects and control subject obtained at a specific stage of the menstrual cycle provide a greater separation between diagnostic groups and higher sensitivity and specificity in distinguishing MDD in females. Therefore, comparison of binding potentials obtained at a specific stage of the menstrual cycle is useful for the diagnosis of depressive disorders in female subjects.

### Example 3

### Diagnosis of Depression in Male Subject

A PET radioligand was injected into the bloodstream of a male subject. One or more PET scans were performed on the male subject. The PET images were analyzed and a receptor binding potential of the PET radioligand at serotonin 5-HT_{1A} receptors in the raphe nuclei of the male subject was determined. The receptor binding potential was determined to be greater than 39.9 and the male subject was classified as having major depression.

A PET radioligand was injected into the bloodstream of a male subject. One or more PET scans were performed the male subject. The PET images were analyzed and a receptor binding potential of the PET radioligand at serotonin 5-HT_{1A} receptors in the raphe nuclei of the male subject was determined. The receptor binding potential was determined to be about the same or less than 39.9 and the male subject was classified as not having major depression.

### Example 4

### Risk for Developing Depression

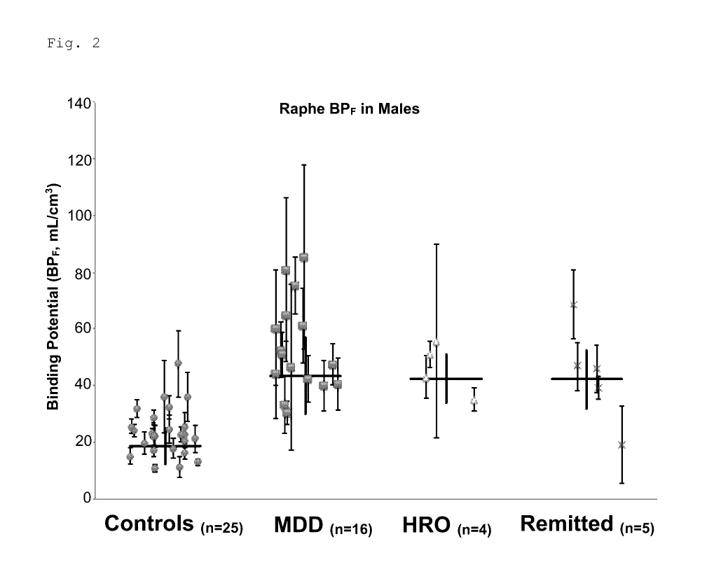

Based on the data presented in FIG. 2, the threshold used for distinguishing male depressed from controls (raphe BP_{F}=39 mL/cm³) also distinguishes male high risk offspring (HRO) from controls. HRO offspring are individuals who have at least one parent diagnosed with depression. Therefore this technology may be used to identify those at risk, while they are currently asymptomatic.

While the best method to separate depressed from controls is currently based on binding potential, it is possible that surrogate markers are available. For example, using just the free fraction only to separate male MDD from male controls with a specificity of 56% or higher. Free fraction measurement or other such surrogate markers also identify those at risk and those currently depressed.

A PET radioligand is injected into the bloodstream of a male subject. One or more PET scans are performed on the male subject. The PET images are analyzed and a receptor binding potential of the PET radioligand at serotonin 5-HT_{1A} receptors in the raphe nuclei of the male subject is determined. The receptor binding potential is determined to be greater than 39.9 and the male subject is classified as at risk for developing major depression

A PET radioligand is injected into the bloodstream of a male subject. One or more PET scans are performed on the male subject. The PET images are analyzed and a receptor binding potential of the PET radioligand at serotonin 5-HT_{1A} receptors in the raphe nuclei of the male subject is determined. The receptor binding potential is determined to be about the same or less than 39.9 and the male subject is classified as not at risk for developing major depression.

### Discussion

Using the PET radioligand N-(2-(4-(2-methoxyphenyl)-1-piperazinyl) ethyl)-N-(2-pyridinyl)cyclo-hexanecarboxamide ([carbonyl-¹¹C]-WAY-100635), a selective 5-HT_{1A} antagonist, it was previously shown that there are higher 5-HT_{1A} binding potential (BP_{F}) in MDD subjects compared to control subjects across several key regions of interest (ROIs) (Parsey et al., 2006; Sullivan et al., 2009). Further, it has been shown that there are differences in the serotonergic system based on sex. In vivo, the mean rate of serotonin synthesis in males was estimated to be 52% higher than in females (Nishizawa et al., 1997). And, postmortem human studies have revealed sex differences in serotonin metabolite 5-hydroxyindole-3-acetic acid (5-HIAA) levels (Gottfries et al., 1974). Using [¹¹C]WAY-100635, it was reported that healthy control women had higher 5-HT_{1A} receptor binding compared to men (Parsey et al., 2002). In 2008, a similar study in controls found that compared to men, women had significantly higher 5-HT_{1A} receptor binding potentials in various cortical and subcortical regions (Jovanovic et al., 2008). 5-HT_{1A} receptor binding in each sex were examined separately using subjects from previously published cohorts (Miller et al., 2009; Parsey et al., 2010; Parsey et al., 2006) to determine whether it would provide more insight into MDD pathophysiology and potentially give rise to a biomarker of the illness.

Both animal and human data show that there are substantial differences in the serotonergic system, and 5-HT_{1A} modulation, between the sexes (Jovanovic et al., 2008). As early as 1970, it was shown that central serotonin levels as well as cerebrospinal fluid concentrations of the serotonin metabolite 5-hydroxyindole-3-acetic acid (5-HIAA) were higher in female than male rats. Further, animal literature has shown that 5-HT_{1A} binding is modulated by estrogen levels in females (Flugge et al., 1999; Frankfurt et al., 1994; Maswood, 1995; Pecins-Thompson & Bethea, 1999; Zhang et al., 1999) and that 5-HT_{1A} autoreceptors in the dorsal raphe nucleus are negatively regulated by female sex hormones (Birzniece et al., 2001; Maswood, 1995; Pecins-Thompson & Bethea, 1999). Conversely, rat studies have shown that estrogen induces up-regulation of 5-HT_{1A} receptors in forebrain regions such as the medial preoptic area (Frankfurt et al., 1994). Finally, it was seen that along with female sex hormones, androgens such as testosterone are able to increase the firing activity of 5-HT neurons in both male and female rats (Robichaud & Debonnel, 2005).

The role of serotonin in depression and, in particular, the relationship between raphe 5-HT_{1A} and MDD (Kishi et al., 2013; Miller et al., 2009; Miller et al., 2013; Parsey et al., 2010; Parsey et al., 2006; Stockmeier et al., 1998), suggests that differences in MDD pathophysiology between the sexes may be due to sex hormones and their modulation of the 5-HT_{1A} receptor. This is confirmed by both animal and human studies (though the majority of animal studies looking at the involvement of 5-HT_{1A} in MDD have used animal cohorts consisting of males only or using a sample with a disproportionate ratio of male to female subjects (Castro et al., 2003; Le Poul et al., 2000; Nishi et al., 2009)). Female ovarian sex hormones, estrogen and progesterone, have been shown to be connected to modulation of mood (Pecins-Thompson & Bethea, 1999). Further, a study looking at the effect of tricyclic antidepressants (TCAs) in depressed rats exposed to chronic stress showed that TCA modulation of 5-HT_{1A} mRNA transcription occurred in a sex dependent manner. Although TCA administration modulated 5-HT_{1A} mRNA expression in the cornu ammonis 1 (CA1) sub-region of the hippocampus, with hippocampal 5-HT_{1A} modulation thought to be involved in MDD, positive TCA effects were only seen in the male rats (Pitychoutis et al., 2012).

In humans, the decline in these sex steroids that occur in females during childbirth and menopause have been correlated with negative affects including depression (Gitlin & Pasnau, 1989), with hormone replacement therapy (e.g. transdermal estrogen) alleviating the depression in some of these subjects (Gregoire et al., 1996). Due to evidence such as this, one common hypothesis put forth by several groups is that depression following female sex hormone decreases is due to changes in the serotonin system within the brain causes by the changes in sex hormone levels (Eriksson et al., 1995; Parry et al., 1993; Halbreich & Tworek, 1997; Su et al. 1997).

It then follows that sex hormones should affect SSRI response, which occurs through the raphe 5-HT_{1A} autoreceptor. These autoreceptors act to inhibit 5-HT postsynaptic neuronal release (Sprouse & Aghajanian, 19$6). Sustained administration of 5-HT_{1A} agonists or SSRIs induces the internalization of 5-HT_{1A} autoreceptors in the raphe nucleus of the midbrain, but not of the post-synaptic hetero-receptors found in the hippocampus (Banerjee et al., 2007). Once this receptor internalization occurs, the efficacy of SSRIs is thought to lie in the fact that the subsequent lack of autoreceptor inhibition allows increased serotonin to be released and bind post-synaptically to 5-HT_{1A} hetero-receptors, therefore inducing the anxiolytic and antidepressant effects of SSRIs (Banerjee et al., 2007).

To date, few studies have looked at the role of sex in the modulation of 5-HT_{1A} specifically in MDD using PET in humans. However, a 2010 study showed that serotonergic differences exist in both healthy individuals and those with MDD across sexes. This study examined alpha-[(11)C]MTrp brain trapping, which is an index of serotonin synthesis (Frey et al., 2010). Sex differences in serotonin synthesis were seen in multiple regions of the prefrontal cortex and limbic system, which are involved in mood regulation. Another study looking into sex differences within the serotonergic system showed that although healthy females exhibit lower cortical trapping of alpha-[(11)C]MTrp than healthy males, females with MDD exhibit higher alpha-[(11)C]MTrp than males with MDD (Frey et al., 2010; Sakai et al., 2006). The current study found a similar trend in that while healthy i.e. control females exhibit higher 5-HT_{1A} binding than healthy males, females with MDD exhibit lower 5-HT_{1A} binding than males with MDD.

Several additional human PET studies have shown that individuals with Bipolar Depression (BD) and MDD exhibit higher 5-HT_{1A} BP_{F} using the radioligand [carbonyl-C-11]-WAY-100635 (Parsey et al., 2006; G. M. Sullivan et al., 2009), and that healthy females exhibit higher 5-HT_{1A} BP_{F} than men. Furthermore, post-hoc analysis in the BD study reported that among the male subjects both the main effect of diagnosis and the region by diagnosis interaction terms were statistically significant.

A possible implication of this study's findings is that previously published reports on the separation of MDD and healthy controls, based on 5-HT_{1A} binding, were potentially driven by the males (Parsey et al., 2010; Parsey et al., 2006). However, this is not to suggest that there are no differences between control and MDD subjects within the female cohort. Rather, similar to the previous preclinical and clinical studies cited above, the results suggest that the pathogenesis of MDD between the two sexes may be different, although the prevalence may be equal when accounting for the different ways in which men and women experience depression.

5-HT_{1A} RN BP_{F} as a Biomarker for MDD Diagnosis

Using neuroimaging, several possible endophenotypes for several psychopathologies have been proposed and include: amygdala/hippocampal volumes in borderline personality disorder via the use of (Ruocco, 2012), brain and CSF volumes in Alzheimer's disease (Reitz, 2009), and white matter pathology and brain volumetric differences in bipolar disorder (Borgwardt, 2012; Hajek, 2005). There even exist multiple potential non-imaging based markers for diagnosing MDD i.e. growth factors, cytokines and endocrine factors, however these markers are limited by a lack of sensitivity and specificity and have not be translated into clinical practice.

This study found that using a threshold value to categorize subjects as either MDD or control based solely on raphe 5-HT_{1A} BP_{F} values yielded extremely high diagnostic sensitivity and specificity (87.5% and 96%, respectively). Using elevated 5-HT_{1A} as a biomarker or endophenotype of MDD could significantly advance our understanding of this psychopathology in several ways. As pointed out by Peterson et al (Peterson, 2011), a biomarker for MDD could aid: in classifying the great heterogeneity observed across MDD presentation into identifiable sub-diagnostic categories and therefore allow more customized treatment strategies; the search for genetic and environmental factors; in identifying those likely to have a chronic course, be treatment resistant, or respond to medication vs. therapy; and in identifying those at increased risk for MDD. The last possibility is especially important since MDD is only 31-42% genetically determined. For this reason, clinicians cannot accurately predict who will develop the illness based only on family history (P. F. Sullivan et al., 2000). Being able to quantify their risk of developing MDD, would allow for preventative strategies to be taken to improve their future mental health outcomes. The current MDD diagnostic criteria do not provide insight into these questions to the extent that a biomarker would.

For these reasons, using 5-HT_{1A} BP_{F} in the raphe as a biomarker for MDD is an extremely promising concept. However, one confound to the practicality of PET in clinical psychiatry is the invasiveness of the procedure i.e. the need of arterial cannulas to calculate BP_{F} binding measures. The free fraction (f_{P}) of a PET tracer, measured from a single venous sample (Milak, 2010a) is used in the calculation of BPF. If the f_{P} value for [Carbonyl-C-11]WAY-100635 was directly correlated with 5-HT_{1A} BP_{F} in the RN, it could be surrogate biomarker for BPF, allowing diagnosis of MDD with a simple venous sampling. However, it was found that [Carbonyl-C-1 l]WAY-100635 f_{P} values achieve much lower specificity than BP_{F} in the raphe (56%).

It is not unreasonable to use PET for depression screening/diagnosis since, identifying those who have MDD, or are at risk for MDD, as early as possible will save time and money in diagnosis and early intervention. However, if this technique were extended to clinical use, it would be greatly beneficial to use additional 5-HT_{1A} PET tracers.

Raphe BP_{F} can be calculated using the tracer [¹¹C]CUMI-101, a 5-HT_{1A} partial agonist. This tracer allows for the BP_{F} estimates to be calculated without the need for blood sampling and the insertion of an arterial cannula (Hendry, 2011; Milak, 2008, 2010b). An F-18 version of [¹¹C]CUMI-101 is also used as a tracer. Due to its 109-minute half-life, an F-18 version of this PET tracer can be shipped to other research centers and does not require a cyclotron on site in order to make the radiotracer. In contrast, compounds made with C-11 have a half-life of only 20 minutes.

### SUMMARY

Currently, there are no diagnostic tests one can perform to determine if an individual is clinically depressed. The clinician must make this determination based on the patient's self-report and the clinician's judgment. This subjective system is prone to error and is also unrelated to the biological causes of depression.

Using 5-HT_{1A} BP_{F} in the raphe as a biomarker for MDD is an extremely promising concept for the field of psychiatry. Although PET technology is currently expensive, it is still used millions of times each year in a variety of medical conditions. Being able to identify those who have MDD, or are at risk for MDD, as early as possible saves downstream time and money in diagnosis and early intervention.

Positron Emission Tomography (PET) is a technique used to visualize and quantify targets in the brain such as neuroreceptors. It is a tool that can be used to understand the disruption of neurotransmitter systems that may occur in depression and other neurological and psychiatric disorders. The systems visualized by PET are dependent on the radiotracer used, and the accuracy of the PET quantification is dependent on the techniques used to analyze the resulting PET images.

Serotonin 5-HT_{1A} binding was found to be higher in healthy females than in healthy males. However, in depressed subjects, serotonin 5-HT_{1A} binding was higher in the males than females. Further analysis focused on serotonin 5-HT_{1A} binding in the raphe, a small region in the midbrain from which most serotonergic neurons originate, in males. Raphe BP_{F} reveals significant differences between male depressed (higher binding) and control subjects. In fact, if a diagnostic threshold of BP_{F} 39.9 mL/cm³ is used, male depressed and control subjects can be distinguished with 96% specificity and 87.5% sensitivity. As such, this method could be the first objective diagnostic test for clinical depression.

### REFERENCES

Ashbumer, J., & Friston, K. J. (1999). Nonlinear spatial normalization using basis functions. Hum Brain Mapp, 7(4), 254-266.
Ashburner, J., & Friston, K. J. (2005). Unified segmentation. Neuroimage, 26(3), 839-851.
Banerjee, P., Mehta, M., & Kanjilal, B. (2007). The 5-HT_{1A} Receptor: A Signaling Hub Linked to Emotional Balance. In A. Chattopadhyay (Ed.), Serotonin Receptors in Neurobiology. Boca Raton (Fla.).
Beck, A. T., Ward, C. H., Mendelson, M., Mock, J., & Erbaugh, J. (1961). An inventory for measuring depression. Arch Gen Psychiatry, 4, 561-571.
Birzniece, V., Johansson, I. M., Wang, M. D., Seckl, J. R., Backstrom, T., & Olsson, T. (2001). Serotonin 5-HT(1A) receptor mRNA expression in dorsal hippocampus and raphe nuclei after gonadal hormone manipulation in female rats. Neuroendocrinology, 74(2), 135-142.
Blier, P., Pineyro, G., el Mansari, M., Bergeron, R., & de Montigny, C. (1998). Role of somatodendritic 5-HT autoreceptors in modulating 5-HT neurotransmission. Ann N Y Acad Sci, 861, 204-216.
Boldrini, M., Underwood, M. D., Mann, J. J., & Arango, V. (2008). Serotonin-1A autoreceptor binding in the dorsal raphe nucleus of depressed suicides. J Psychiatr Res, 42(6), 433-442.
Borgwardt, S. & Fusar-Poli, P. (2012). White matter pathology—an endophenotype for bipolar disorder? BMC Psychiatry, 12, 138.
Brix, G., et al. (1997) Performance evaluation of a whole-body PET scanner using the NEMA protocol. National Electrical Manufacturers Association. J Nucl Med 38: 1614-1623.
Castro, M., Diaz, A., del Olmo, E., & Pazos, A. (2003). Chronic fluoxetine induces opposite changes in G protein coupling at pre and postsynaptic 5-HT_{1A} receptors in rat brain. Neuropharmacology, 44(1), 93-101.
Cleare A J & Bond A J. (2000) Experimental evidence that the aggressive effect of tryptophan depletion is mediated via the 5-HT_{1A} receptor. Psychopharmacology (Berl) 147: 439-441
Drevets, W. C., et al. (1999). PET imaging of serotonin 1A receptor binding in depression. Biol Psychiatry, 46(10), 1375-1387.
Endicott, J., Spitzer, R. L., Fleiss, J. L., & Cohen, J. (1976). The global assessment scale. A procedure for measuring overall severity of psychiatric disturbance. Arch Gen Psychiatry, 33(6), 766-771.
Eriksson E.; Hedberg M. A.; Andersch B. and Sundblad. (1995). The serotonin reuptake inhibitor paroxetine is superior to the noradrenaline reuptake inhibitor maproltiline in the treatment of premenstrual syndrome. Neuropsychopharmacology, 12, 167-176.
Flugge, G. et al. (1999). 5-HT_{1A}-receptor binding in the brain of cyclic and ovariectomized female rats. J Neuroendocrinol, 11(4), 243-249.
Frankfurt, M., McKittrick, C. R., Mendelson, S. D., & McEwen, B. S. (1994). Effect of 5,7-dihydroxytryptamine, ovariectomy and gonadal steroids on serotonin receptor binding in rat brain. Neuroendocrinology, 59(3), 245-250.
Frey, B. N. et al. (2010). Gender differences in alpha-[(11)C]MTrp brain trapping, an index of serotonin synthesis, in medication-free individuals with major depressive disorder: a positron emission tomography study. Psychiatry Res, 183(2), 157-166.
Gitlin M. J.; Pasnau R. (1989). Psychiatric syndromes linked to reproductive function in women: a review of current knowledge. American Journal of Psychiatry, 146, 1413-1422.
Gottfries, C. G. et al. (1974). Determination of 5-hydroxytryptamine, 5-hydroxyindoleacetic acid and homovanillic acid in brain tissue from an autopsy material. Acta Psychiatr Scand, 50(5), 496-507.
Gregoire, A. J., Kumar, R., Everitt, B., Henderson, A. F., & Studd, J. W. (1996). Transdermal oestrogen for treatment of severe postnatal depression. Lancet, 347(9006), 930-933.
Gunn R N, Sargent P A, Bench C J, Rabiner E A, Osman S, Pike V W, Hume S P, Grasby P M, Lammertsma A A: Tracer kinetic modeling of the 5-HT_{1A} receptor ligand [carbonyl-11C]WAY-100635 for PET. Neuroimage 1998, 8(4):426-440.
Hajek, T., Carrey, N. & Alda, M. (2005). Neuroanatomical abnormalities as risk factors for bipolar disorder. Bipolar Disord, 7(393-403).
Halbreich U.; Tworek. (1993). Altered serotonergic activity in women with dysphoric premenstrual syndromes. Int. J. Psychiat. Med., 23, 1-27.
Hamilton, M. (1960). A rating scale for depression. J Neurol Neurosurg Psychiatry, 23, 56-62.
Hendry, N., Christie, I., Rabiner, E. A., Laruelle, M. & Watson, J. (2011). In vitro assessment of the agonist properties of the novel 5-HT(1A) receptor ligand, CUMI-101 (MMP), in rat brain tissue. Nucl Med Biol, 38, 273-277.
Huang, Y. Y. et al. (2004). Human 5-HT_{1A} receptor C(-1019)G polymorphism and psychopathology. Int J Neuropsychopharmacol, 7(4), 441-451.
Hwang, D. R., et al. (1999) An improved one-pot procedure for the preparation of [11C-carbonyl]-WAY100635. Nucl Med Biol 26: 815-819.
Innis, R. B. et al. (2007). Consensus nomenclature for in vivo imaging of reversibly binding radioligands. J Cereb Blood Flow Metab, 27(9), 1533-1539.
Jenkinson, M., & Smith, S. (2001). A global optimisation method for robust affine registration of brain images. Med Image Anal, 5(2), 143-156.
Jovanovic, H. et al. (2008). Sex differences in the serotonin 1A receptor and serotonin transporter binding in the human brain measured by PET. Neuroimage, 39(3), 1408-1419.
Karisson, L. et al. (2010). Minor change in the diagnostic threshold leads into major alteration in the prevalence estimate of depression. J Affect Disord, 122(1-2), 96-101.
Kishi, T. et al. (2013). The serotonin 1A receptor gene confer susceptibility to mood disorders:
results from an extended meta-analysis of patients with major depression and bipolar disorder. Eur Arch Psychiatry Clin Neurosci, 263(2), 105-118.
Le Poul, E. et al. (2000). Differential adaptation of brain 5-HT_{1A} and 5-HT_{1B} receptors and 5-HT transporter in rats treated chronically with fluoxetine. Neuropharmacology, 39(1), 110-122.
Lopez, A. D., & Murray, C. C. (1998). The global burden of disease, 1990-2020. Nat Med, 4(11), 1241-1243.
Marangell, L. B. et al. (2006). Prospective predictors of suicide and suicide attempts in 1,556 patients with bipolar disorders followed for up to 2 years. Bipolar Disord, 8, (5 Pt 2), 566-575.
Maswood, S.; Stewart, G.; Uphouse, L. (1995). Gender and oestrus cycle effects of the 5-HT_{1A} agonist 8-OH-DPAT on hypothalamic serotonin. Pharmacol Biochem Behav, 51, 807-813.
Merikangas, K. R. et al. (2007). Lifetime and 12-month prevalence of bipolar spectrum disorder in the National Comorbidity Survey replication. Arch Gen Psychiatry, 64(5), 543-552.
Martin, L. A., Neighbors, H. W., & Griffith, D. M. (2013). The experience of symptoms of depression in men vs women: analysis of the National Comorbidity Survey Replication. JAMA Psychiatry, 70(10), 1100-1106.
Milak, M. S. et al. (2008). Modeling considerations for 11C-CUMI-101, an agonist radiotracer for imaging serotonin 1A receptor in vivo with PET. J Nucl Med, 49, 587-596.
Milak, M. S. et al. (2010a). In vivo quantification of human serotonin 1A receptor using 11C-CUMI-101, an agonist PET radiotracer. Journal of Nuclear Medicine, 51, 1892-1900.
Milak, M. S. et al. (2010b). In vivo quantification of human serotonin 1A receptor using 11C-CUMI-101, an agonist PET radiotracer. J Nucl Med, 51, 1892-1900.
Miller, J. M. et al. (2009). Elevated serotonin 1A binding in remitted major depressive disorder: evidence for a trait biological abnormality. Neuropsychopharmacology, 34(10), 2275-2284.
Miller, J. M. et al. (2013). Brain Serotonin 1A Receptor Binding as a Predictor of Treatment Outcome in Major Depressive Disorder. Biol Psychiatry, 74(10), 760-767.
Nishi, K. et al. (2009). A genetic rat model of depression, Flinders sensitive line, has a lower density of 5-HT(1A) receptors, but a higher density of 5-HT(1B) receptors, compared to control rats. Neurochem Int, 54(5-6), 299-307.
Nishizawa, S. et al. (1997). Differences between males and females in rates of serotonin synthesis in human brain. Proc Natl Acad Sci USA, 94(10), 5308-5313.
Ogden, R. T., & Tarpey, T. (2006). Estimation in regression models with externally estimated parameters. Biostatistics, 7(1), 115-129.
Parker, G., & Brotchie, H. (2010). Gender differences in depression. Int Rev Psychiatry, 22(5), 429-436.
Parry B. (1997). Psychobiology of premenstrual dysphoric disorder. Semin. Reprod. Endocr., 15, 55-68.
Parsey, R. V. et al. (2010). Higher serotonin 1A binding in a second major depression cohort: modeling and reference region considerations. Biol Psychiatry, 68(2), 170-178.
Parsey, R. V. et al. (2006). Altered serotonin 1A binding in major depression: a [carbonyl-C-11]WAY100635 positron emission tomography study. Biol Psychiatry, 59(2), 106-113.
Parsey, R. V. et al. (2005). Regional heterogeneity of 5-HT_{1A} receptors in human cerebellum as assessed by positron emission tomography. J Cereb Blood Flow Metab, 25(7), 785-793.
Parsey, R. V., Oquendo, M. A., Simpson, N. R., Ogden, R. T., Van Heertum, R., Arango, V., & Mann, J. J. (2002). Effects of sex, age, and aggressive traits in man on brain serotonin 5-HT_{1A} receptor binding potential measured by PET using [C-11]WAY-100635. Brain Res, 954(2), 173-182.
Parsey, R. V. et al. (2000). Validation and reproducibility of measurement of 5-HT_{1A} receptor parameters with [carbonyl-¹¹C]WAY-100635 in humans: comparison of arterial and reference tissue input functions. J Cereb Blood Flow Metab, 20(7), 1111-1133.
Pecins-Thompson, M., & Bethea, C. L. (1999). Ovarian steroid regulation of serotonin-1A autoreceptor messenger RNA expression in the dorsal raphe of rhesus macaques. Neuroscience, 89(1), 267-277.
Reitz, C. & Mayeux, R. (2009). Endophenotypes in normal brain morphology and Alzheimer's disease: a review. Neuroscience, 164, 174-190.
Robichaud, M., & Debonnel, G. (2005). Oestrogen and testosterone modulate the firing activity of dorsal raphe nucleus serotonergic neurones in both male and female rats. J Neuroendocrinol, 17(3), 179-185.
Ruocco, A. C., Amirthavasagam, S. & Zakzanis, K. K. (2012). Amygdala and hippocampal volume reductions as candidate endophenotypes for borderline personality disorder: a meta-analysis of magnetic resonance imaging studies. Psychiatry Res, 201, 245-252.
Pitychoutis, P. M., Dalla, C., Sideris, A. C., Tsonis, P. A., & Papadopoulou-Daifoti, Z. (2012). 5-HT(1A), 5-HT(2A), and 5-HT(2C) receptor mRNA modulation by antidepressant treatment in the chronic mild stress model of depression: sex differences exposed. Neuroscience, 210, 152-167.
Sakai, Y. et al. (2006). Cortical trapping of alpha-[(11)C]methyl-1-tryptophan, an index of serotonin synthesis, is lower in females than males. Neuroimage, 33(3), 815-824.
Sargent, P. A. et al. (2000). Brain serotonin 1A receptor binding measured by positron emission tomography with [11C]WAY-100635: effects of depression and antidepressant treatment. Arch Gen Psychiatry, 57(2), 174-180.
Savitz, J., Lucki, I., & Drevets, W. C. (2009). 5-HT(1A) receptor function in major depressive disorder. Prog Neurobiol, 88(1), 17-31.
Singh, I., & Rose, N. (2009). Biomarkers in psychiatry. Nature, 460(7252), 202-207.
Smith, S. M. (2002). Fast robust automated brain extraction. Hum Brain Mapp, 17(3), 143-155.
Sprouse J. S.; Aghajanian G. (1986). Propanolol blocks the inhibition of serotonergic dorsal raphe cell firing by 5-I-ITIA selective agonists. Eur. J. Pharmac., 128, 295-298.
Stockmeier, C. A. (2003). Involvement of serotonin in depression: evidence from postmortem and imaging studies of serotonin receptors and the serotonin transporter. J Psychiatr Res, 37(5), 357-373.
Su T. P. et al. (1997). Effect of menstrual cycle phase on neuroendocrine and behavioral responses to the serotonin agonist m-chlorophenylpiperazine in women with premenstrual syndrome and controls. J. Clin. Endocr. Metab., 82, 1220-1228.
Sullivan, G. M. et al. (2009). Positron emission tomography quantification of serotonin-1A receptor binding in medication-free bipolar depression. Biol Psychiatry, 66(3), 223-230.
Sullivan, P. F. et al. (2000). Genetic epidemiology of major depression: review and meta-analysis. Am J Psychiatry, 157, 1552-1562.
Woods, S. W. (2000). The economic burden of bipolar disease. J Clin Psychiatry, 61 Supp 13, 38-41.
Wu, S., & Comings, D. E. (1999). A common C-1018G polymorphism in the human 5-HT_{1A} receptor gene. Psychiatr Genet, 9(2), 105-106.
Zhang, L. et al. (1999). Sex differences in expression of serotonin receptors (subtypes 1A and 2A) in rat brain: a possible role of testosterone. Neuroscience, 94(1), 251-259.
Zhang, Y., et al. (2001) Segmentation of brain MR images through a hidden Markov random field model and the expectation—maximization algorithm. IEEE Trans Med Imaging 20: 45-57.
World Health Organization (2004) The global burden of disease: 2004 update.

1. A method of determining whether a subject is afflicted with a depressive disorder comprising:
(i) introducing into the subject a positron emission tomography (PET) radioligand capable of binding with a serotonin 5-HT_{1A} receptor;
(ii) performing one or more PET scans of the subject;
(iii) determining, by analysis of the one or more PET images, a receptor binding potential of the PET radioligand for the serotonin 5-HT_{1A} receptor in a region of interest in the subject;
(iv) comparing the receptor binding potential value of the PET radioligand in the region of interest in the subject to a predetermined receptor binding potential threshold value; and
(v) classifying the subject as having the depressive disorder or as not having the depressive disorder based on the comparison of step (iv), thereby determining whether the subject is afflicted with the depressive disorder.

2. The method of claim 1, wherein the PET radioligand is introduced by injection into the bloodstream of the subject.

3. The method of claim 1, wherein the analysis of the one or more PET images in step (iii) is a computer analysis.

4. The method of claim 1, wherein step (ii) further comprises carrying out one or more MRI scans of the subject.

5. The method of claim 4, wherein the MRI images are analyzed to define the boundaries of the region of interest.

6. The method of claim 1, wherein the subject is classified as having the depressive disorder when the receptor binding potential value of the PET radioligand in the region of interest in the subject is greater than the predetermined diagnostic threshold value.

7. The method of claim 1, wherein the subject is classified as not having the depressive disorder when the receptor binding potential value of the PET radioligand in the region of interest in the subject is about the same or less than the predetermined diagnostic threshold value.

8. The method of claim 1, wherein the PET radioligand contains a radioisotope selected from the group consisting of ³H, ¹¹C, ¹³N, ¹⁸F, ¹²³I, ¹²⁵I, ^{99m}Tc, ⁹⁵Tc, ¹¹¹In, ⁶²Cu, ⁶Cu, ⁴⁴Sc ⁶⁷Ga, and ⁶⁸Ga.

9. The method of claim 8, wherein the PET radioligand contains an ¹¹C radioisotope or an ¹⁸F radioisotope.

10. (canceled)

11. The method of claim 9, wherein the PET radioligand is radiolabeled N-[2-[4-(2-methoxyphenyl)-1-piperazinyl]ethyl]-N-2-pyridinylcyclohexanecarboxamide or radiolabeled 2-(4-(4-(2-methoxyphenyl)piperazin-1-yl)butyl)-4-methyl-1,2,4-triazine-3,5 (2H,4H)dione.

12.-17. (canceled)

18. The method of claim 1, wherein the region of interest is in the brain.

19. The method of claim 18, wherein the region of interest is selected from the group consisting of the raphe nucleus, dorsolateral prefrontal cortex, medial prefrontal cortex, orbito-frontal cortex, anterior cingulate cortex, subgenual prefrontal cortex, temporal cortex, parietal cortex, occipital cortex, amygdala, uncus, hippocampal formation, entorhinal cortex, parahippocampal gyrus, insula, dorsal raphe nuclei, and cerebellum.

20. (canceled)

21. The method of claim 20, wherein the predetermined receptor binding potential threshold value is 39.9.

22. The method of claim 1, wherein the depressive disorder is major depression.

23. The method of claim 1, wherein the subject is a human subject.

24. The method of claim 23, wherein the human subject is a male subject.

25. The method of claim 24, wherein the male subject had never undergone antidepressant treatment; or the male subject had gone without antidepressant treatment for at least four years.

26. (canceled)

27. A method of preparing a report classifying a subject as having a depressive disorder or as not having a depressive disorder which comprises:
(i) receiving the data of one or more PET scans of the subject performed by a PET imaging device after a PET radioligand for a serotonin 5-HT_{1A} receptor was introduced into the subject;
(ii) processing the data to determine a receptor binding potential of the PET radioligand for the serotonin 5-HT_{1A} receptor in a region of interest in the subject and comparing the receptor binding potential value to a predetermined receptor binding potential threshold value; and
(iii) populating a report classifying the subject.

28. A method of treating a subject afflicted with a depressive disorder, comprising
(a) determining whether the subject is afflicted with the depressive disorder comprising:
(i) introducing into the subject a positron emission tomography (PET) radioligand capable of binding with a serotonin 5-HT_{1A} receptor;
(ii) performing one or more PET scans of the subject;
(iii) determining, by analysis of the one or more PET images, a receptor binding potential of the PET radioligand for the serotonin 5-HT_{1A} receptor in a region of interest in the subject;
(iv) comparing the receptor binding potential value of the radioligand in the region of interest in the subject to a predetermined receptor binding potential threshold value; and
(v) classifying the subject as afflicted with the depressive disorder when the receptor binding potential value of the radioligand in the subject is greater than the predetermined diagnostic threshold value; and
(b) treating the subject based on the determination obtained in step (a).

29.-31. (canceled)

32. The method of claim 28, wherein the subject is treated with an anti-depressant or psychotherapy.

33.-35. (canceled)
